(19) United States
(12) Patent Application Publication
(10) Pub. No.: US 2015/0231097 A1
(43) Pub. Date: Aug. 20, 2015

(54) COMPOUND TO PREVENT OR TREAT CONTACT DERMATITIS CAUSED BY POISONOUS PLANTS

(71) Applicant: Alexa Blake, Whitmore Lake, MI (US)

(72) Inventor: Alexa Blake, Whitmore Lake, MI (US)

(21) Appl. No.: 14/627,712

(22) Filed: Feb. 20, 2015

Related U.S. Application Data
(60) Provisional application No. 61/942,440, filed on Feb. 20, 2014.

(51) Int. Cl. A61K 31/19; A61K 31/56; A61K 9/00; A61K 31/045

(52) U.S. Cl. CPC A61K 31/19; A61K 31/045; A61K 31/56; A61K 9/0014

(57) ABSTRACT

Poisonous plants release urushiol, an oil that can cause dermatitis or irritation of the skin. A compound to alleviate symptoms of a poison plant rash is disclosed. The compound an anti-itch agent to soothe the skin and prevent scratching and a chemical peel agent to remove the urushiol from the skin. The compound can optionally further include alcohol or a hand sanitizer.

### CROSS REFERENCE TO RELATED APPLICATIONS

This disclosure is claims the benefit of U.S. Provisional Application No. 61/942,440 filed on Feb. 20, 2014 which is hereby incorporated by reference.

### TECHNICAL FIELD

This disclosure is related to the use of a compound to prevent or treat contact dermatitis of human skin caused by contact with compounds contained within certain poisonous plants.

### BACKGROUND

The statements in this section merely provide background information related to the present disclosure. Accordingly, such statements are not intended to constitute an admission of prior art.

Many plants are poisonous to humans, especially when poisonous plant chemicals come in contact with skin. The most common problems with poisonous plants arise from contact with the sap oil of plants that cause an allergic skin reaction: poison ivy, poison oak, and poison sumac. These three plants are distributed widely throughout the United States.

Poison ivy, poison oak, and poison sumac release an oil, urushiol, when the leaf or other plant parts are bruised or otherwise damaged. When the urushiol comes in contact with the skin, an allergic reaction called contact dermatitis occurs in most exposed people as an itchy red rash with bumps or blisters. When exposed to 50 micrograms of urushiol, 80 to 90 percent of adults will develop a rash. Signs and symptoms associated with dermal contact with poisonous plants may include: red rash within a few days of contact; bumps, patches, streaking, or weeping blisters; swelling; and itching. The fluid from the weeping blisters does not contain urushiol and contact with this fluid does not cause contact dermatitis in others.

People may become exposed to urushiol directly through contact with the urushiol-containing plant, or indirectly through contact with tools, livestock, or clothing that have urushiol on them. Urushiol tends to adhere to whatever surface it comes in contact with, and can remain active on the surface of objects for up to 5 years unless the objects are thoroughly cleaned with rubbing alcohol (isopropanol or isopropyl alcohol).

### SUMMARY

Poisonous plants release urushiol, an oil that can cause dermatitis or irritation of the skin. A compound to alleviate symptoms of a poison plant rash is disclosed. The compound an anti-itch agent to soothe the skin and prevent scratching and a chemical peel agent to remove the urushiol from the skin. The compound can optionally further include alcohol or a hand sanitizer.

### BRIEF DESCRIPTION OF THE DRAWINGS

One or more embodiments will now be described, by way of example, with reference to the accompanying drawings, in which:

FIG. 1 illustrates the application of an embodiment of the class of disclosed compounds to an affected area of human skin that has previously come into contact with urushiol from a poisonous plant such as poison ivy, poison oak, or poison sumac, in accordance with the present disclosure.

### DETAILED DESCRIPTION

Referring now to the drawings, wherein the showings are for the purpose of illustrating certain exemplary embodiments only and not for the purpose of limiting the same, a composition is disclosed including an anti-itch agent and a skin peel agent. The anti-itch agent is provided to calm the skin and prevent the patient from scratching and irritating the skin. The skin peel agent removes a top layer of the skin. The oils from the poisonous plant permeate a surface of the skin, and as long as the oils remain close to the skin, the allergic reaction in the skin continues to irritate the skin. By applying the skin peel, a majority of the oil is removed with the top layer of the skin thereby removing the irritant.

An anti-itch agent is provided to prevent the user from scratching the affected area. Topical anti-itch agents belong to a class of drugs called antipruritics. Antipruritics include a number of specific compounds. Many fall within the sub-classes antihistamines, corticosteroids, local anesthetics, and counter-irritants. Scratching can spread any remaining urushiol to other areas and cause the irritation to spread. Further, scratching can damage the skin and lead to infection and further irritation of the affected area.

A compound to treat exposure to a poisonous plant is further provided including a chemical peel or an acid skin peel (for example, including lactic acid or glycolic acid). By removing a top layer of the epidermis, skin that was exposed to and may still be saturated with some of the urushiol is removed. A compound including rubbing alcohol and a chemical peel is disclosed in order to remove the irritant from the area of the skin and remove a top layer of skin to further prevent traces of the irritant from further irritating the area of the patients skin. Acids used for skin peels are known to include particular effective concentrations, and the disclosure is intended to include concentrations known for use in approved products. In one exemplary skin peel product, the effective acid is permitted in a 3% to 7% concentration. The disclosed compound is constituted of ingredients which do not interfere with the skin peel properties of the skin peel agent, for example, with the pH of the compound being controlled.

In some embodiments, rubbing alcohol can be used to relieve the rash caused by poison ivy, poison oak, and poison sumac. Rubbing alcohol is a generic term for ethyl alcohol, isopropyl alcohol, or any other similar substance, and references to rubbing alcohol herein are intended to refer to any alcohol substance known in the art that can be applied to skin of a patient. This relief or cure may result from the hand sanitizer's action as a solvent to break down the urushiol, or the hand sanitizer's action as a cleansing agent that removes the urushiol from the pores of the skin. Removing the irritant can prevent or alleviate the dermatitis associated with the irritant. It should be noted that any type of alcohol approved for use in a topical / medical ointment, such as common grain alcohol, can be used with the disclosed composition.

Rubbing alcohol can be found as a standalone product, or rubbing alcohol can be contained in a composition or compound with other ingredients. One embodiment of a compound that can include rubbing alcohol is hand sanitizers. Embodiments of the present disclosure can include hand sanitizer known in the art with additional ingredients disclosed herein. Other embodiments of the present disclosure include rubbing alcohol with additional ingredients disclosed herein in a liquid form or a lotion in a state that would not be commonly considered a hand sanitizer. A number of alternative compounds including the ingredients disclosed herein are envisioned, and the disclosure is not intended to be limited to the particular examples provided.

One example of a hand sanitizer may include the following ingredients: ethyl alcohol (70%), water, isopropyl alcohol, PEG-12 dimethicone, caprylyl glycol, glycerin, isopropyl myristate, tocopheryl acetate and fragrance. Another example of a hand sanitizer may include the following ingredients: alcohol, water, glycerin, dimethicone, petrolatum, aminomethylpropanol, panthenol, hydroxypropyl cellulose, ceteth-10, steareth-21, poloxamer 335 and aloe vera leaf. Another example of a hand sanitizer may include the following: benzalkonium chloride, dihydroxypropyl PEG-5 linoleammonium chloride, glycereth-2 cocoate, behentrimonium chloride and dihydroxyethyl cocamine oxide. These ingredient lists are provided as non-limiting examples, and it will be appreciated that any sanitizer or compound with similar ingredients are envisioned within the disclosure.

A compound can benefit from including all three of rubbing alcohol, an anti-itch agent, and a chemical peel agent. Removing the urushiol oil from the skin, removing a top layer of skin with remaining traces of the urushiol oil, and preventing the patient from itching the affected area, a patient can prevent or quickly recover from a rash caused by exposed to urushiol oil.

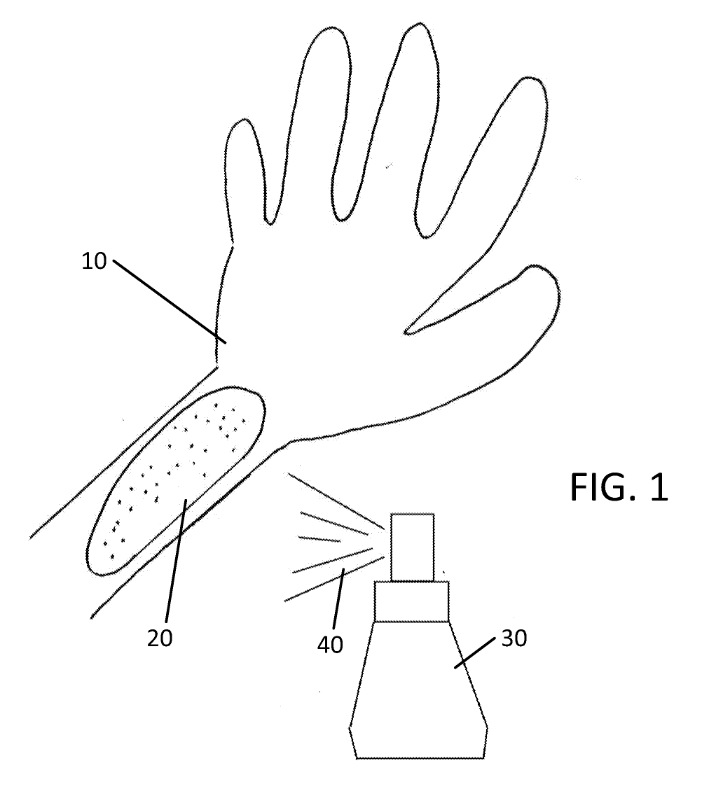

FIG. 1 illustrates the application of hand sanitizer to an affected area of human skin that has previously come into contact with urushiol from a poisonous plant such as poison ivy, poison oak, or poison sumac. A patient's arm 10 is illustrated including an affected area 20. An exemplary spray bottle 30 is illustrated including a spray 40 applying the disclosed compound to the affected area. Spray bottle 30 can be substituted with any application device or method known in the art.

Such a compound as disclosed herein can advantageously be sprayed upon the skin, for example, by a bottle utilizing a spray/atomizer pump device. In the alternative, the compound can be applied by an absorbent pad device, as a lotion, or any other delivery device or method known in the art.

The disclosure has described certain preferred embodiments and modifications of those embodiments. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

1. A compound to alleviate symptoms of a poison plant rash, the compound comprising:
an anti-itch agent; and
a chemical peel agent.

2. The compound of claim 1, wherein the chemical peel agent comprises lactic acid.

3. The compound of claim 1, wherein the chemical peel agent comprises glycolic acid

4. The compound of claim 1, further comprising alcohol.

5. The compound of claim 1, further comprising hand sanitizer.

6. The compound of claim 1, wherein the compound is constituted to be sprayed upon skin of a patient.

7. The compound of claim 1, wherein the anti-itch agent comprises an antihistamine.

8. The compound of claim 1, wherein the anti-itch agent comprises a corticosteroid.

9. The compound of claim 1, wherein the anti-itch agent comprises a local anesthetic.

10. The compound of claim 1, wherein the anti-itch agent comprises a counter-irritant.
